(19) United States
(12) Patent Application Publication
(10) Pub. No.: US 2003/0199797 A1
(43) Pub. Date: Oct. 23, 2003

(54) Plaster cast sheath

(76) Inventor: Olarewaju J. Oladipo, Canton, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/405,916

(22) Filed: Apr. 2, 2003

(30) Foreign Application Priority Data
Apr. 2, 2002 (GB) GB 0207547.1

(51) Int. Cl. A61F 5/05

(52) U.S. Cl. 602/11; 602/5; 602/22

(57) ABSTRACT

A plaster cast sheath for use during the application of a cast to an extremity of a patient is disclosed. The plaster cast sheath allows for the correct placement of a portion of the unwinding roll of plaster cast material as it is passed through the interdigital web space, e.g., between the thumb and index finger or the big toe and second toe, or between any two digits on the extremity that needs to be immobilized, as the cast is being prepared on the extremity. The plaster cast sheath is used for the purpose of protecting the skin that lies on either side of the band of plaster material that passes through the limited interdigital space between two adjacent digits of the hand or foot.

### CROSS REFERENCE TO RELATED APPLICATIONS

N/A

This application claims the priority of Great Britain Application No. GB 0207547.1 filed Apr. 2, 2002 entitled, PLASTER CAST SHEATH, the whole of which is hereby incorporated by reference herein.

### STATEMENT REGARDING FEDERALLY SPONSORED RESEARCH OR DEVELOPMENT

N/A

### BACKGROUND OF THE INVENTION

Casts are commonly used in the treatment of sprains, fractures and wounds in the extremities. These casts could be made, e.g., of Plaster of Paris, fiberglass or polyester substrate impregnated with a polymer that becomes activated and hardens when dipped in water. In the use of these materials on a wrist, for example, a strip of the plaster material is passed over the base of the interdigital web space between the thumb and the index finger in order to secure the cast to the hand of the patient. The same situation sometimes arise after surgery to the foot, requiring the placement of a strip of plaster cast between the big and second toes. Usually, more than one layer of the strip is inserted in the limited space between two digits. To allow for the placement of multiple strips, the widths of the strips are reduced with a pair of scissors.

As the polymer in the plaster material is activated, it hardens, with some of the material exerting pressure on the skin below the layer of padding under the plaster and on the area of skin on either side of the web space. A common complication when a cast is applied is the onset of abrasions to the surrounding skin, with the added risk of infection.

This is particularly a problem in children, diabetic patients, and patients with compromised circulation in their extremities. For example, geriatric patients or patients who are confused may not observe the progressive ulceration and infection of the skin. In a few cases, simple abrasions stemming from a cast applied to an extremity have resulted in deep ulceration, secondary infection and gangrene in the uninjured extremity. Parts of the body commonly affected include the first interdigital webspace of the foot, between the big and second toes, and the space between the thumb and index finger.

There are, therefore, obvious limitations to the currently available options. Accordingly, there is a need for a better way to protect these vulnerable parts of the body.

### BRIEF SUMMARY OF THE INVENTION

Accordingly, an object of this invention is to provide a means of protecting the interdigital web spaces of the body from accidental injury that may occur from the abrasive surface of an applied cast.

According to one aspect of the present invention, the foregoing and other objects and advantages are attained by the use of a plaster cast sheath that is placed over the area of padding that is usually applied directly to a patient's skin as the first layer during the process of cast application. The sheath of the invention serves as a tunnel sleeve for a strip or strips of plaster cast material passing in the interdigital web space between two adjacent digits so as to protect the adjacent areas of skin during and following cast application. The plaster cast sheath of the invention comprises an elongated body of malleable consistency usually having a hollow cavity or groove to receive the unwound strip of plaster cast passing through the interdigital web space of two adjacent digits.

The plaster cast sheath of the invention, which may be in any desired shape, may have one or more wing-like flanges attached to its external wall or incorporated into its body. The flanges are molded against the skin bordering the interdigital web space. The cavity of the cast sheath may also be subdivided by longitudinal ridges on the inner portion of the side wall(s) to serve for placement of individual strips of plaster cast material.

Other objects, advantages and novel aspects of the present invention will become apparent in the detailed description of the invention that follows, taken in conjunction with the accompanying drawings, in which the preferred embodiment is shown by way of illustration of the best mode contemplated for carrying out the invention.

### BRIEF DESCRIPTION OF THE SEVERAL VIEWS OF THE DRAWINGS

FIG. 1 is a perspective view of a plaster cast sheath incorporating the principles of the invention;

FIG. 2A is a perspective view of another embodiment of a plaster cast sheath according to the invention; and

FIG. 2B is a perspective view of an additional embodiment of a plaster cast sheath according to the invention.

### DESCRIPTION OF THE PREFERRED EMBODIMENT OF THE INVENTION

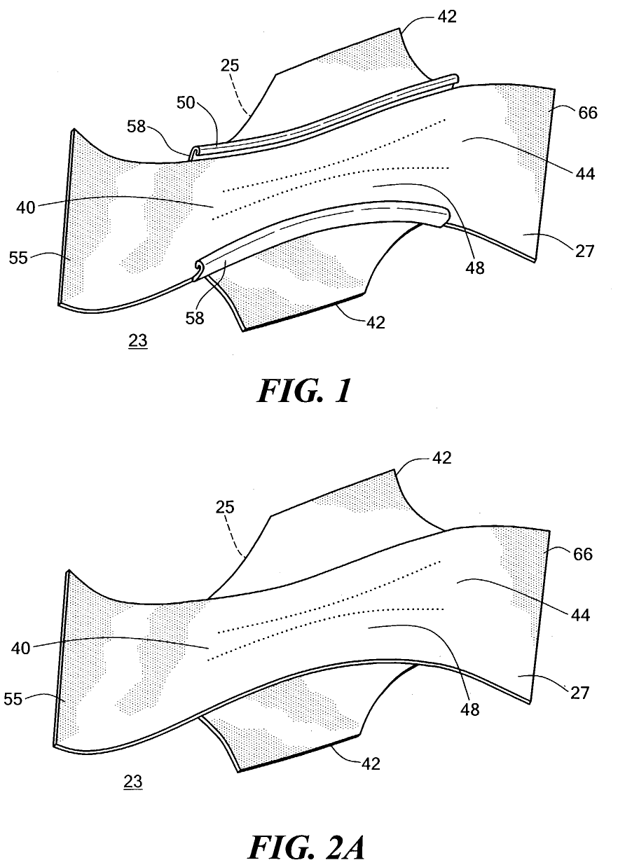

As shown in FIG. 1, a preferred embodiment of a plaster cast sheath according to the invention includes an elongated body 23 of malleable consistency having a proximal end 55 and a distal end 66. The two ends are connected by a middle, or bridge, segment 44, which may be grooved or may consist of a hollow cavity to receive the unwinding strip of plaster cast material as it is passed through the interdigital web space of two adjacent digits. The plaster cast sheath has an interior surface 25, the side of the sheath that is positioned toward the skin of the patient, and an exterior surface 27. In one embodiment of this invention, the plaster cast sheath is made with a longitudinal opening or cavity 40 along part of the length of the exterior surface 27. Cavity 40 is bordered by two side walls 58, each topped by a rolled edge 50. The elongated body 23 is preferably narrower in width in the middle section and wider at the proximal and distal ends. The ends of the tubular body flare out such the tubular enclosure becomes flat at each end. This allows the strip of plaster cast material to overlap these flattened edges as it is passed through the interdigital web space, e.g., between the thumb and index finger, and over, e.g., the wrist area. The rolled edges 50 of the body form the border of tubular enclosure 40.

The plaster cast sheath elongated body 23 may have one or more wing-like flanges 42 attached to its external wall. Flanges 42 may be separately attachable or may be incorporated into the cast sheath during its manufacture, depending on the design of a specific sheath embodiment. These flanges are made of a flexible material and are trimmed or molded to fit against the skin bordering the interdigital web space. The hollow cavity may also be subdivided by longitudinal ridges 48 on the inner wall to serve for placement of individual strips of plaster cast material.

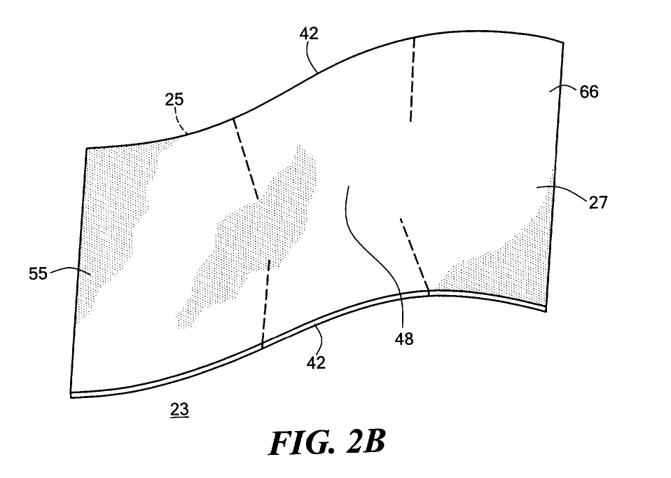

Referring now to FIG. 2A, in another embodiment of the invention, the plaster cast sheath is flat on exterior surface 27, instead of having a longitudinal opening, with one or more flanges 42 on its sides to protect the skin against the sharp edges of the hardened plaster material. A version of FIG. 2A that is very simple to manufacture is shown in FIG. 2B. In this embodiment, the cast sheath has the general shape of an undulating rectangle, to fit over the curvature of the interdigital web space, e.g. between the thumb and index fingure of the left hand. Flanges 42 are die cut so that they can be raised away from the main body of the cast sheath, to protect the digits on either side of the web space.

The inner surface 25 of the contoured body is preferably smooth, and because it is made of a flexible material, it is easily molded to fit over the skin between two adjacent digits. The plaster cast sheath is preferably made of a combination of one or more of the commercial padding materials, e.g., wool, neoprene, polyurethane, silicone, polyvinyl or any other suitable material.

To use the plaster cast sheath of the invention during cast application, the appropriate size and shape is chosen for the site of application and the sheath is fitted into the interdigital web space over a layer of cotton padding applied as the first layer during plaster cast application. As the strip of plaster cast is unwound and applied in the interdigital groove, the strips are positioned through the longitudinal groove or opening on the superior wall of the sheath. The groove surface or longitudinal opening serves as a tunnel, preventing skin contact when the cast material has been set. The diameter and shape of the plaster cast sheath used depends on the size of the web space and desired number of plaster strips to be inserted.

When desired, the interior surface of the plaster cast sheath is coated with an adhesive and protected with a removable liner to allow the sheath to stay on the surface of the underlying padding until the plaster strips have been applied.

This described invention provides a reliable and user friendly way to protect the skin of the interdigital web space of the hand or foot from the abrasive and/or pressure effects of a portion of a cast that must pass through the space between two adjacent digits.

While the present invention has been described with reference to a preferred embodiment thereof, illustrated in the accompanying drawings, various changes and modifications can be made by those skilled in the art without departing from the spirit and scope of the present invention; therefore, the appended claims are to be construed to cover equivalent structures.

What is claimed is:

1. A plaster cast sheath for the protection of the skin of the interdigital web space of an extremity of a patient during cast application, said cast sheath comprising:
an elongated substrate composed of a material having a malleable consistency for fitting said cast sheath in the interdigital web space of two adjacent digits, said substrate having a proximal end, a distal end and a middle section, wherein said proximal and distal ends are substantially flat and wherein the edges of said middle section are capable of being elevated with respect to the center portion of said middle section, said substrate further having an interior surface, which is positioned toward the skin of the patient when the sheath is in use, and an exterior surface, which is positioned away from the skin of the patient when the sheath is in use.

2. The cast sheath of claim 1, wherein said edges of said middle section comprise one or more flanges along the sides of said substrate and wherein said flanges are moldable for fitting against the skin bordering the interdigital web space.

3. The cast sheath of claim 1, further comprising longitudinal ridges in the middle section of said substrate for aiding placement of individual strips of plaster cast material during cast preparation.

4. The cast sheath of claim 1, wherein said proximal and said distal ends of said substrate are wider than the middle section of said substrate.

5. The cast sheath of claim 1, wherein said middle section of said substrate comprises a longitudinal cavity bordered by longitudinally oriented sidewalls.

6. The cast sheath of claim 1, wherein the interior surface of said substrate is coated with an adhesive which is protected with a removable liner.
